(19) United States
(12) Patent Application Publication
(10) Pub. No.: US 2026/0368883 A1
(43) Pub. Date: Nov. 19, 2026

(54) APPARATUS AND METHOD FOR PREDICTING DEEP LEARNING-BASED HIP JOINT LESION

(71) Applicants: SEOUL NATIONAL UNIVERSITY HOSPITAL, Seoul (KR); UNIVERSITY OF ULSAN FOUNDATION FOR INDUSTRY COOPERATION, Ulsan (KR)

(72) Inventors: Jung-Wee PARK, Seoul (KR); Young-Kyun LEE, Seoul (KR); Jeong Joon YOO, Seoul (KR); Hong Seok KIM, Seoul (KR); Seung Min RYU, Seoul (KR)

(21) Appl. No.: 19/136,866

(22) PCT Filed: Dec. 15, 2023
(86) PCT No.: PCT/KR2023/020771

§ 371 Date: Jan. 17, 2026

(30) Foreign Application Priority Data
Dec. 15, 2022 (KR) 10-2022-0176188

(51) Int. Cl. G06T 7/00; G06V 10/72; G06V 10/764; G06V 10/774; G06V 10/82

(52) U.S. Cl. CPC G06T 7/0012; G06V 10/72; G06V 10/764; G06V 10/774; G06T 2207/10116; G06T 2207/20081; G06T 2207/20084; G06T 2207/30008; G06T 2207/30096; G06V 10/82; G06V 2201/033

(57) ABSTRACT

A hip joint lesion prediction apparatus according to an embodiment comprises: one or more processors; and a memory that stores a program executed by the one or more processors, wherein the one or more processors: acquire a target radiographic image by capturing a hip joint, and output whether or not the hip joint is abnormal in the target radiographic image by applying the radiographic image to a deep learning model.

### TECHNICAL FIELD

The disclosed embodiments relate to an apparatus and a method for predicting a hip joint lesion, and more particularly to a technique for predicting a hip joint lesion using deep learning.

### CROSS-REFERENCE TO RELATED APPLICATION

This application claims priority to Korean Patent Application No. 10-2022-0176188 filed on Dec. 15, 2022, the entire contents of which are incorporated herein by reference.

### BACKGROUND ART

Hip joint lesions are primarily diagnosed by plain hip radiography. Plain radiography, e.g., X-ray, is widely used, unlike greater-level tests, such as computed tomography (CT) and magnetic resonance imaging (MRI).

However, it is not an easy task for medical staff at a primary hospital who encounter plain radiographs on the medical front line to discriminate between hip joint diseases. For patients suffering from various diseases, such as a kidney disease, a spinal disease, a urological disease, and a gynecological disease, in addition to hip joint pain, plain radiography focusing on the hip joint is performed.

If a hip joint disease is not found even after plain radiography, the hip joint may be destroyed due to deformation of the femoral head or loss of articular cartilage with reduced joint spacing of the hip joint. Total hip arthroplasty, which involves inserting artificial joints into the acetabulum and femur, is a surgical option but it is a final measure. It is most preferable to detect a hip joint disease in advance through initial detection.

Therefore, for a hip joint disease, medical staff in fields who do not mainly deal with radiographic images also need screening knowledge. Hip joint lesions should be diagnosed through an automated model without reading by orthopedic surgeons and radiologists who mainly deal with radiographic images.

### DETAILED DESCRIPTION OF THE INVENTION

### Technical Solution

The disclosed embodiments are intended to provide an apparatus and a method for predicting a hip joint lesion.

### SUMMARY OF THE INVENTION

A hip joint lesion prediction method according to an embodiment is a method performed by a deep learning-based hip joint lesion prediction apparatus including one or more processors; and a memory that stores one or more programs executed by the one or more processors, the method including: acquiring a target radiographic image by capturing a hip joint; and outputting whether or not a hip joint is abnormal in the target radiographic image by using a deep learning model.

The number of layers, the number of filters, and the size of the input image of the deep learning model may be determined as interdependent and associated values.

The number of layers, the number of filters, and the size of the image of the deep learning model may be determined as values having common exponents and associated with each other.

In the deep learning model, an output of a previous layer may be connected to an input of all subsequent layers.

In the deep learning model, a feature map of a previous layer may be connected to a feature map of a next layer.

The deep learning model may include one or more non-local blocks for global learning on the input image.

The deep learning model is learned based on a hip joint radiographic image classified according to electronic medical records (EMR) medical information extracted using a clinical data warehouse (CDW). The hip joint radiographic image may be labeled with at least one of a normality and a diagnosis.

The hip joint radiographic image may be preprocessed such that at least one of a character, a symbol, a mark, and a pixel with a preset brightness value or greater included in the medical information is removed.

A hip joint lesion prediction apparatus according to an embodiment includes: one or more processors; and a memory that stores a program executed by the one or more processors, wherein the one or more processors: acquire a target radiographic image by capturing a hip joint, and output whether or not the hip joint is abnormal in the target radiographic image by applying the radiographic image to a deep learning model.

The number of layers, the number of filters, and the size of the input image of the deep learning model may be determined as interdependent and associated values.

The number of layers, the number of filters, and the size of the image of the deep learning model may be determined as values having common exponents and associated with each other.

In the deep learning model, an output of a previous layer may be connected to an input of all subsequent layers.

In the deep learning model, a feature map of a previous layer may be connected to a feature map of a next layer.

The deep learning model may include one or more non-local blocks for global learning on the input image.

The deep learning model is learned based on a hip joint radiographic image classified according to electronic medical records (EMR) medical information extracted using a clinical data warehouse (CDW), and the hip joint radiographic image may be labeled with at least one of a normality and a diagnosis.

The hip joint radiographic image may be preprocessed such that at least one of a character, a symbol, a mark, and a pixel with a preset brightness value or greater included in the medical information is removed.

### Effects of Invention

The disclosed embodiments may assist in predicting or diagnosing a hip joint disease with high sensitivity and specificity for a radiographic image taken of a hip joint through a deep learning-based hip joint prediction model.

### BRIEF DESCRIPTION OF THE DRAWINGS

FIG. 1 is a block diagram of a hip joint lesion prediction apparatus according to an embodiment.

FIG. 2 is an exemplary diagram of target radiographic images of an example.

FIG. 3 is an exemplary diagram of target radiographic images of an example.

FIG. 4 is an exemplary diagram illustrating a structure of a deep learning model.

FIG. 5 is a graph illustrating the performance of the hip joint lesion prediction apparatus for each embodiment.

FIG. 6 is a flowchart of a hip joint lesion prediction method according to an embodiment.

### BEST MODE FOR CARRYING OUT THE INVENTION

Hereinafter, specific embodiments are described with reference to the drawings. The following detailed description is provided to facilitate a comprehensive understanding of a method, an apparatus, and/or a system described herein. However, this is merely an example and the present invention is not limited thereto.

In describing the embodiments, when it is determined that the detailed description of the known art related to the present invention may unnecessarily obscure the gist of the embodiment, the detailed description thereof will be omitted. In addition, the terms described below are terms defined in consideration of the functions in the present invention, which may vary depending on the intention or practice of a user, an operator, or the like. Therefore, the definition should be made based on the content throughout this specification. The terms used in the detailed description are only for the purpose of describing one embodiment without limiting the same. Unless specifically used otherwise, the singular forms include the meaning of plural forms. In the present description, expressions such as “comprising” or “including” are intended to refer to certain components, numbers, steps, operations, elements, parts or combinations thereof, and should not be construed to exclude the presence or possibility of one or more other components, numbers, step, operation, elements, parts, or combinations thereof other than those described.

Furthermore, embodiments described herein may have aspects that are entirely hardware, partially hardware and partially software, or entirely software. In this specification, a “unit”, a “module”, a “device”, a “server”, or a “system” refers to hardware, a combination of hardware and software, or a computer-related entity such as software. For example, the unit, the module, the device, the server, or the system may refer to hardware constituting part or all of a platform and/or software, such as an application for driving the hardware.

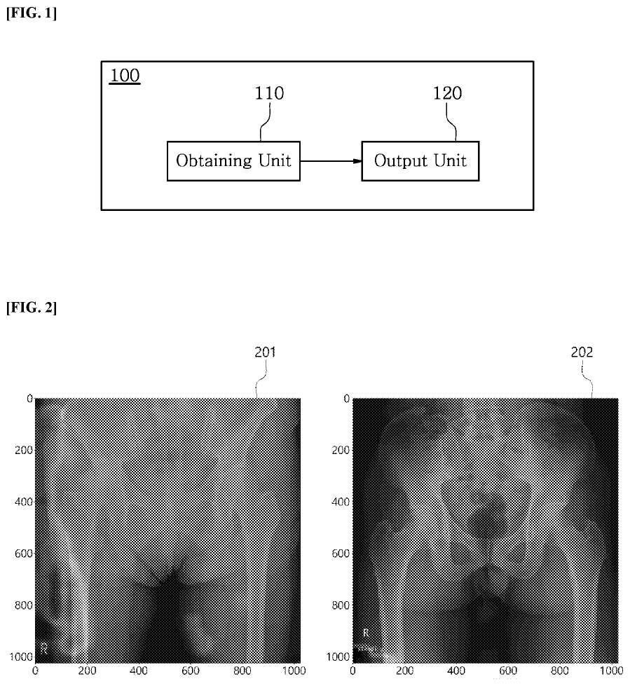

FIG. 1 is a block diagram of a hip joint lesion prediction apparatus 100 according to an embodiment.

Referring to FIG. 1, the hip joint lesion prediction apparatus 100 according to an embodiment includes an acquisition unit 110 and an output unit 120.

The acquisition unit 110 and the output unit 120 may be implemented by using one or more devices that are physically separated or may be implemented by one or more processors or a combination of one or more processors and software, and may not be clearly separated in specific operations, unlike the illustrated example.

The acquisition unit 110 acquires a target radiographic image by capturing a hip joint.

The target radiographic image may refer to an image taken by a medical device that is universally accessible in a primary hospital as a plain radiograph. The target radiographic image may be, for example, an X-ray image.

The output unit 120 outputs whether or not the hip joint is abnormal in the target radiographic image by using the deep learning model.

The output unit 120 may output an abnormality classification result of the hip joint with respect to the target radiographic image by using the deep learning model. Specifically, the output unit 120 may display and output a categorical variable (normal, abnormal) as a vector based on one-hot encoding.

The output unit 120 may output a classification result of the hip joint lesion with respect to the target radiographic image by using the deep learning model.

For example, the output unit 120 may output a classification result of whether or not the hip joint has at least one of amyloidosis, arthritis, osteonecrosis of femoral head (ONFH), and hip joint tumor in the target radiographic image by using the deep learning model.

The number of layers, the number of filters, and the size of the input image of the deep learning model may be determined as interdependent and associated values.

The deep learning model may simultaneously scale three parameters including the number of layers, the number of filters, and the size of the input image to determine the value of the loss function to be small. In this case, the three parameters may be interdependent and associated values.

The deep learning model may uniformly scale the three parameters including the number of layers, the number of filters, and the size of the input image to determine the value of the loss function to be small. In this case, the three parameters may be interdependent and associated values.

For example, the deep learning model may scale the three parameters including the number of layers, the number of filters, and the size of the input image to have a positive correlation with each other.

For example, the deep learning model may scale such that all three parameters including the number of layers, the number of filters, and the size of the input image are proportional to each other.

For example, the deep learning model may scale such that at least one pair of parameters among the number of layers, the number of filters, and the size of the input image are proportional to each other.

For example, the deep learning model may scale such that at least one pair of parameters among the number of layers, the number of filters, and the size of the input image are inversely proportional to each other.

For example, the deep learning model may scale such that at least one pair of parameters among the number of layers, the number of filters, and the size of the input image are proportional to each other, and at least the other pair of parameters are inversely proportional to each other.

The number of layers, the number of filters, and the size of the image of the deep learning model may be set to values that are fixed and associated with each other.

The number of layers, the number of filters, and the size of the image of the deep learning model may be scaled to values having common exponents and associated with each other. In this case, the values for the number of layers, the number of filters, and the size of the image may be constants determined based on the lattice search. In other words, the values for the number of layers, the number of filters, and the number of images may be determined based on a constant showing the highest performance after sequentially inputting any number specified in the dictionary.

The highest performance may mean when the loss function value of the deep learning model becomes the smallest.

The loss function may include, for example, at least one of a cross-entropy loss function and a binary cross-entropy loss function.

The deep learning model may scale the number of layers, the number of filters, and the size of the input image based on Equation 1 below.

$$\{ \begin{matrix}{L}_{n} = {a}^{x} \\ {F}_{n} = {b}^{x} \\ R = {c}^{x} \\ {a}^{x} * {b}^{x} * {c}^{x} < A\end{matrix} \qquad [ Equation 1 ]$$

Lₙ is the number of layers, Fₙ is the number of filters, and R is the size of the image; a, b, and c are any real numbers greater than 0, x is any real number, and A is a preset value.

In this way, the deep learning model may improve the performance through more balanced scaling, compared to an existing method of performing random scaling on an arbitrary parameter.

The deep learning model may be configured to directly connect all layers to each other.

Specifically, the deep learning model may be configured to connect the output of the previous layer to the input of the next layer.

Specifically, the deep learning model may be configured to connect each layer output value to an input value of all layers subsequent to the corresponding layer.

The all layers are layers included in a specific block, and the deep learning model may include one or more blocks so that all layers for each block may be directly connected to each other.

The deep learning model may be configured to concatenate a feature map of the previous layer with a feature map of the next layer.

In this case, the resolutions of the feature map of the previous layer and the feature map of the next layer may be the same or different from each other.

The feature map of the previous layer is a feature map for a layer included in a specific block. The deep learning model may include one or more blocks so that feature maps of all layers for each block may be concatenated with feature maps of subsequent layers. The blocks may be bundled for each layer with the same resolution of the feature maps.

As such, the deep learning model can prevent loss of previously extracted features or input values by reusing the feature maps of the previous layers.

Thus, the deep learning model prevents loss of the feature maps of the previous layers, thereby reducing the degree of weight freedom.

That is, the deep learning model may exert an effect similar to a regularization effect where overfitting is canceled by reducing weight freedom.

The number of layers, the number of filters, and the size of the image of the deep learning model may be limitedly scaled within a preset range by multiplying the values of the number of layers, the number of filters and the size of the image of the deep learning model. In this case, the preset range may include the memory of the deep learning model.

The deep learning model may further include a first downsampling layer that adjusts the number of channels of the extracted feature map.

The deep learning model may further include a second downsampling layer that lowers the resolution of the extracted feature map.

As such, the deep learning model may perform operations with small parameters through the first and second downsampling layers.

As such, the deep learning model may allow the use of feature maps of various resolutions through the second downsampling layer.

The deep learning model may include at least one non-local block for global learning on the input image. The non-local block may include a convolution filter of 1*1 size for learning all pixels of the image.

This allows the deep learning model to capture long-range dependences by weighting the values of the features present at locations in the image using the non-local block.

The deep learning model may be learned based on a hip joint radiographic image included in electronic medical records (EMR) medical information extracted using a clinical data warehouse (CDW).

The hip joint radiographic image may be labeled with at least one of a diagnosis and a normality of the hip joint.

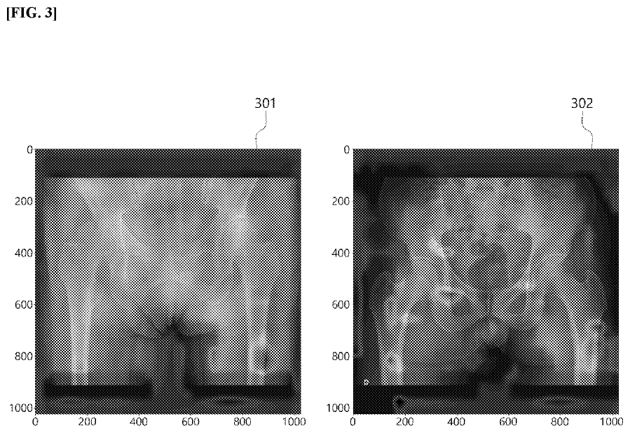

Specifically, the hip joint radiographic image may be an X-ray image by capturing a hip joint labeled with at least one of a normality and a diagnosis, which is primarily diagnosed by an imaging medicine department and secondarily diagnosed by a hip joint specialist. The hip joint radiographic image may be raw data of the X-ray image in FIG. 2 or a preprocessed X-ray image in FIG. 3 to be described below.

In a non-limiting embodiment, when the deep learning model is learned based on, for example, a total of 1000 hip joint X-ray images, 500 hip joint X-ray images may be disease images taken of a diseased hip joint. Specifically, the disease image may be a hip joint radiographic image of a patient diagnosed with a hip joint disease by an imaging medicine department and a hip joint specialist. Specifically, the disease image may be a hip joint X-ray image of a hip joint disease patient last taken before being treated with artificial hip joint prosthesis.

The remaining 500 hip joint X-ray images may be normal images taken of a disease-free hip joint. Specifically, the normal image may be a hip joint X-ray image of a patient diagnosed as normal by an imaging medicine department and a hip joint specialist with respect to the hip joint.

In this case, the deep learning model may be learned by dividing training data, verification data, and test data in a ratio of 64:16:20 for a total of 1000 datasets. For example, the deep learning model may divide 640 training data into 10 mini-batches and learn at least 30 epochs.

The deep learning model, which is a model based on a backbone network, may be, for example, a model based on ResNet, VGGNet, or DarkNet. The backbone network may refer to a network including an encoding process of compressing an input image into images of various channels and a process of extracting features using a convolution operation during the encoding process.

The hip joint radiographic image may be information preprocessed such that at least one of a character, a symbol, a mark, and a pixel with a preset brightness value or greater included in the hip joint radiographic images is removed.

The hip joint radiographic image may be information preprocessed such that one or more first pixels having a brightness value equal to or greater than a first preset value or one or more second pixels having a brightness value equal to or less than a second preset value have a brightness between the first and second values.

Specifically, the hip joint radiographic image may be processed based on data cleaning, image matching, image cropping, image clipping, and the like, to remove factors that interfere with the deep learning model's learning to predict hip joint lesions, such as a character, a symbol, a mark, an artifact, and the like included in the hip joint radiographic image.

On the other hand, even though the information that is preprocessed and removed from the hip joint radiographic image has been described as a character, a symbol, a mark, an artifact, and the like, this is only an example. The information is intended to be removed without being limited thereto as long as it is a detected object other than the hip joint.

FIG. 2 is an exemplary diagram of target radiographic images of an example.

Referring to FIG. 2, the target radiographic images may include a first target radiographic image 201 or a second target radiographic image 202.

As shown in FIG. 2, the target radiographic images may include the first target radiographic image 201 acquired by capturing an anterior view of the hip joint. Alternatively, the target radiographic images may include the second target radiographic image 202 acquired by capturing a posterior view of the hip joint. Alternatively, the target radiographic images may include a radiographic image (not shown) of a left side view or a right side view of the hip joint. In other words, the target radiographic images may include radiographic images of the hip joint taken at various angles without being limited to the angles.

The target radiographic images may be raw data of the radiographic images taken of the hip joint. In this case, the raw data may include at least one of a capturing date, a capturing time, a medical device logo, a medical device identifier, and an image identifier that are stamped by capturing a radiographic image, which are desirably deleted.

On the other hand, the target radiographic images have been described as being radiographic images acquired by capturing a front surface, a back surface, or side surfaces of the hip joint, but this is an example. The angle at which the target radiographic images are captured is not limited thereto.

FIG. 3 is an exemplary diagram of target radiographic images of an example.

Referring to FIG. 3, the target radiographic images may include a third target radiographic image 301 or a fourth target radiographic image 302.

In this case, the third target radiographic image 301 and the fourth target photographic image 302 may be images acquired by preprocessing the first target radiographic image 201 and the second target photographic image 202 of FIG. 2, respectively.

Specifically, each of the third target radiographic image 301 and the fourth target radiographic image 302 may be an image from which a character, a symbol, a mark, and an artifact included in the first target radiographic image 201 and the second target radiographic image 202 are removed. As a specific example, the third target radiographic image 301 and the fourth target radiographic image 302 may be radiographic images where at least one of a capturing date, a capturing time, a medical device logo, a medical device identifier, and an image identifier stamped on the first target radiographic image 201 and the second target radiographic image 202 is cropped.

The third target radiographic image 301 and the fourth target radiographic image 302 may be radiographic images cropped so as not to include a body part located away from the hip joint. Specifically, the third target radiographic image 301 and the fourth target radiographic image 302 may be radiographic images taken so as to include the entire hip joint, but only a part of the other body organs.

The third target radiographic image 301 and the fourth target radiographic image 302 may be radiographic images cropped so as not to include a part separated by a preset distance from the center of the target radiographic image.

The third target radiographic image 301 and the fourth target radiographic image 302 may be radiographic images that are cropped to shorten a height length of the image. At this time, the edge of the target radiographic image is preferably prioritized for the target region to be cropped. For example, when the third target radiographic image 301 and the fourth target radiographic image 302 are cropped to shorten the height length from the size of the entire image 1024*1024 to 1024*800, the height from the center to the top of the target radiographic image may be 400 and the height from the center to the bottom of the target radiographic image may be 400.

The third and fourth target radiographic images 301 and 302 may be zero padded after cropping to maintain the original pixel size. For example, the target radiographic image of which the pixel size is modified to 1024*800 after cropping may be zero padded to maintain the original pixel size until it reaches the original pixel size 1024*1024.

On the other hand, the target radiographic image has been described as described above with reference to FIG. 2 and FIG. 3, but this is not limited to the target radiographic image. It is intended that the description may also be applied to the learning data of the hip joint lesion prediction apparatus 100.

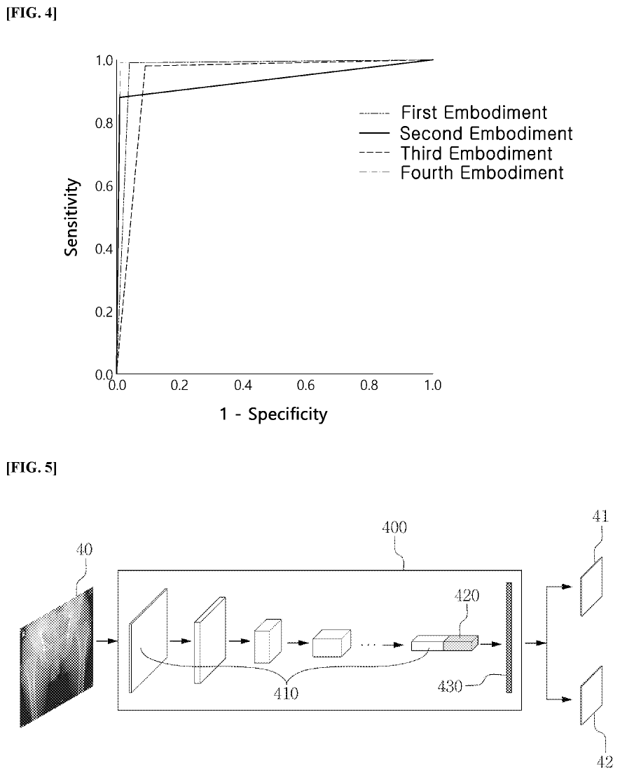

FIG. 4 is an exemplary diagram illustrating a structure of a deep learning model 400.

Referring to FIG. 4, the deep learning model 400 includes one or more convolution blocks 410, a non-local block 420, and a fully-connected layer 430.

Each of the one or more convolution blocks 410 may be designed to include at least one of a convolution layer, an activation function, a batch normalize layer, and a pooling layer.

The convolution layer corresponding to each of the one or more convolution blocks 410 may include a first convolution layer, which is a general convolution layer.

The convolution layer corresponding to each of the one or more convolution blocks 410 may include a second convolution layer that applies a convolution operation by dividing the input image or the feature map for each channel. As such, the second convolutional layer may capture features of the input values with a small amount of parameters.

In addition, the second convolutional layer may compress several channels into one new channel by fixing the size of the filter to 1. Thus, the second convolutional layer may adjust the number of channels to adjust the amount of computation.

The convolution layer corresponding to each of the one or more convolution blocks 410 may include a third convolution layer to which an expansion factor for the number of channels is otherwise applied to the second convolution layer.

The resolution of the image or feature map input to the convolution layer corresponding to each of the one or more convolution blocks 410 may be designed to be less than or equal to the resolution of the image and the feature map input to a convolution layer of a previous stage as the stage deepens.

At this time, the number of convolutional layers included in each of the one or more convolution blocks 410 may be designed to be greater than or equal to the number of convolution layers included in the previous convolution block 410 as the stage deepens.

For example, one or more convolution blocks 410 may be designed as shown in Table 1 below.

TABLE 1

| | | | Number Of |
|---|---|---|---|
| | | | Convolutional |
| Stage | Convolution Layer, Kernel Size | Resolution | Layers |
| 1 | First convolutional layer, 3 × 3 | 300 × 300 | 1 |
| 2 | Second convolutional layer, 3 × 3 | 150 × 150 | 2 |
| 3 | Third convolutional layer, 3 × 3 | 150 × 150 | 3 |
| 4 | Third convolutional layer, 5 × 5 | 75 × 75 | 3 |
| 5 | Third convolutional layer, 3 × 3 | 38 × 38 | 5 |
| 6 | Third convolutional layer, 5 × 5 | 19 × 19 | 5 |
| 7 | Third convolutional layer, 5 × 5 | 10 × 10 | 6 |
| 8 | Third convolutional layer, 3 × 3 | 10 × 10 | 2 |

Specifically, referring to the first column of Table 1, eight convolution blocks 410 may be designed to be sequentially connected to each other. Referring to the second column of Table 1, the convolution block 410 may include at least one of a first convolution layer, a second convolution layer, and a third convolution layer. For example, the convolutional block 410 of the first stage may be designed to include a first convolutional layer, the convolutional block 410 of the second stage may include a second convolutional layer, and the convolutional blocks 410 of the third to eighth stages may include third convolutional layers. In this case, the kernel size may be different or the same for each convolution block 410.

Referring to the third column of Table 1, the resolutions of the images or feature maps input to the convolution block 410 are 300×300, 150×150, 150×100, 75×75, 38×38, 19×19, 10×10, and 10×10, respectively, and the resolutions may be designed to be the same or smaller as the stage deepens. Thus, the deep learning model 400 may perform an encoding process of compressing an input image and extracting a feature.

Referring to the fourth column of Table 1, the number of convolutional layers included in the convolution block 410 may be 1, 2, 3, 3, 5, 5, 6, and 2, respectively, and may be designed to be greater than or equal to the number of convolution layers included in the previous convolution block 410 as the stage deepens.

The non-local block 420 may be connected to the convolution block 410 of the last stage.

The fully-connected layer 430 may be connected to the last of the blocks included in the deep learning model 400 to binary classify the target radiographic image 40 according to the extracted feature. For example, the deep learning model may classify the target radiographic image 40 as normal 41 or abnormal 42 through the fully-connected layer 430.

Meanwhile, the structure of the deep learning model 400 of FIG. 4 is illustrative and not necessarily limited thereto.

On the other hand, the second convolution layer may be a convolution layer based on MBConv1, and the third convolution layer may be a convolution layer based on the MBConv6.

FIG. 5 is a graph illustrating the performance of the hip joint lesion prediction apparatus for each embodiment.

Referring to FIG. 5, a receiver operating characteristic (ROC) curve of each of the first to fourth embodiments of the hip joint lesion prediction apparatus 100 is shown.

The first embodiment may be a hip joint lesion prediction apparatus 100, wherein both the input image and the learning image are raw data of the radiographic image taken of the hip joint, the hip joint lesion prediction apparatus 100 learned and inferred based on the raw data. It is assumed that the hip joint lesion prediction apparatus 100 infers a hip joint lesion based on the first deep learning model.

The first deep learning model may include a model where an output of a previous layer is connected to an input of a next layer.

The first deep learning model may include a model where an output of a previous layer is connected to an input of all subsequent layers.

The first deep learning model may include a deep learning model designed based on a dense-net.

The second embodiment may be a hip joint lesion prediction apparatus 100 that infers a hip joint lesion for an input image where inference elements are cropped, and that is learned based on raw data that is a radiographic image taken of the hip joint. In this case, it is assumed that the hip joint lesion prediction apparatus 100 infers a hip joint lesion based on the first deep learning model.

A third embodiment may be a hip joint lesion prediction apparatus 100 that infers a hip joint lesion for an input image where inference elements are cropped, and where the inference elements have been learned based on the cropped learning image. In this case, it is assumed that the hip joint lesion prediction apparatus 100 infers a hip joint lesion based on the first deep learning model.

A fourth embodiment may be a hip joint lesion prediction apparatus 100 that infers a hip joint lesion for an input image where inference elements are cropped, and where the inference elements have been learned based on the cropped learning image. In this case, it is assumed that the hip joint lesion prediction apparatus 100 infers a hip joint lesion based on the second deep learning model.

The second deep learning model may include a model designed such that the number of layers, the number of filters, and the size of the input image are determined to be interdependent and associated values.

The second deep learning model may include a deep learning model designed based on efficient-net. For example, the second deep learning model may include deep learning designed based on at least one of efficient-net V1-B1 to B7. Preferably, the second deep learning model may include deep learning designed based on efficient-net V1-B3.

The second deep learning model may include at least one non-local block for global learning.

At this time, referring to FIG. 5, the fourth embodiment has greater classification performance than the other embodiments with respect to whether or not the hip joint is abnormal. However, the first to third embodiments have poor classification performance, compared to the fourth embodiment due to the following problems.

Specifically, a problem of learning the inference elements, e.g., a character to be stamped, is found in the first embodiment. A problem of learning by focusing on the entire femur, which is a body part other than the hip joint, is found in the second embodiment. A problem of learning by focusing on a greater sciatic notch is found in the third embodiment.

In addition, other performance indicators of the hip joint lesion prediction apparatus 100 according to each embodiment may be shown in Table 2 below.

TABLE 2

| | First | Second | Third | Fourth |
|---|---|---|---|---|
| | Embodiment | Embodiment | Embodiment | Embodiment |
| Accuracy | 0.98 | 0.93 | 0.95 | 0.99 |
| Sensitivity | 0.99 | 0.88 | 0.98 | 0.99 |
| Specificity | 0.96 | 0.99 | 0.91 | 0.99 |
| Positive | 0.96 | 0.99 | 0.92 | 0.99 |
| Predictive | | | | |
| Value (PPV) | | | | |
| Negative | 0.99 | 0.90 | 0.98 | 0.99 |
| Predictive | | | | |
| Value (NPV) | | | | |

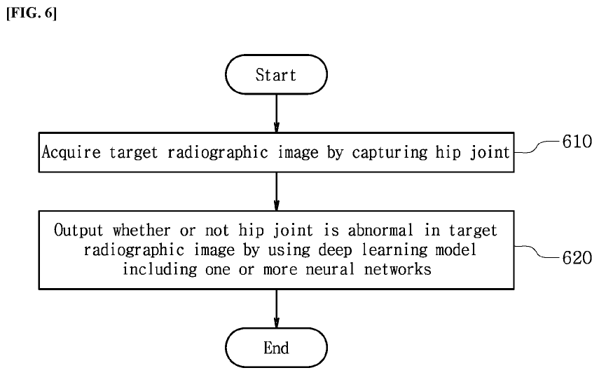

As can be seen from Table 2 above, the fourth embodiment is evaluated to be the best in terms of accuracy, sensitivity, specificity, positive predictive value, and negative predictive value, compared to the other embodiments. FIG. 6 is a flowchart of a hip joint lesion prediction method according to an embodiment.

The hip joint lesion prediction method according to an embodiment of FIG. 6 may be performed by the hip joint lesion prediction apparatus 100 of FIG. 1. First, the hip joint lesion prediction apparatus 100 acquires a target radiographic image by capturing a hip joint (610).

Then, the deep learning model is used to output whether or not the hip joint is abnormal in the target radiographic image (620).

FIG. 6 has been described with reference to the order presented in the drawings. Although the method has been shown and described as a series of blocks for purposes of illustration, the invention is not limited to the order of the blocks. Some blocks may occur concurrently with other blocks or in a different order from that shown and described herein. Various other branches, flow paths, and orders of blocks that achieve the same or similar results may be implemented. In addition, not all blocks shown for implementing the method described herein may be required.

On the other hand, an embodiment of the present invention may include a program for performing the method described herein on a computer, and a computer-readable recording medium including the program. The computer-readable recording medium may include program instructions, local data files, local data structures, and the like, alone or in combination. The medium may be specially designed and configured for the present invention or conventionally usable in the field of computer software. Examples of the computer-readable recording medium includes a magnetic medium, such as a hard disk, a floppy disk, and a magnetic tape, an optical recording medium, such as CD-ROM, a DVD, and a hardware device specifically configured to store and perform program instructions such as ROM, RAM, flash memory, and the like. Examples of such programs may include machine language code, such as that made by a compiler, as well as advanced language code that may be executed by a computer using an interpreter or the like.

While representative embodiments of the present invention have been described above in detail, those skilled in the art will appreciate that various modifications may be made to the above-described embodiments without departing from the scope of the present invention. Therefore, the scope of the present invention should not be limited to the described embodiments but should be defined by the following claims as well as those equivalent to the claims.

### INDUSTRIAL APPLICABILITY

The deep learning-based hip joint lesion prediction apparatus and method according to an embodiment may determine the prognosis of a patient without human intervention by applying a radiographic image of a hip joint to a deep learning model, and thus may be used in the digital medical industry.

1. A deep learning-based hip joint prediction method, performed by a deep learning-based hip joint lesion prediction apparatus having one or more processors and a memory that stores one or more programs executed by the one or more processors, the deep learning-based hip joint prediction method comprising:
acquiring a target radiographic image of the hip joint; and
outputting whether or not the hip joint in the target radiographic image is abnormal by using a deep learning model.

2. The deep learning-based hip joint prediction method of claim 1, wherein the number of layers, the number of filters, and the size of the input image of the deep learning model are determined as interdependent and associated values.

3. The deep learning-based hip joint prediction method of claim 2, wherein the number of layers, the number of filters, and the size of the input image of the deep learning model are determined as values having common exponents and associated with each other.

4. The deep learning-based hip joint prediction method of claim 1, wherein, in the deep learning model, an output of a previous layer is connected to an input of all subsequent layers.

5. The deep learning-based hip joint prediction method of claim 1, wherein, in the deep learning model, a feature map of a previous layer is connected to a feature map of a next layer.

6. The deep learning-based hip joint prediction method of claim 1, wherein the deep learning model comprises one or more non-local blocks for global learning on the input image.

7. The deep learning-based hip joint prediction method of claim 1, wherein the deep learning model is learned based on a hip joint radiographic image classified according to electronic medical records (EMR) medical information extracted using a clinical data warehouse (CDW), and
the hip joint radiographic image is labeled with at least one of a normality and a diagnosis.

8. The deep learning-based hip joint prediction method of claim 7, wherein the hip joint radiographic image is preprocessed such that at least one of a character, a symbol, a mark, and a pixel with a preset brightness value or greater included in the hip joint radiographic image is removed.

9. A hip joint lesion prediction apparatus comprising:
one or more processors; and
a memory configured to store a program executed by the one or more processors, wherein the one or more processors acquire a target radiographic image by capturing a hip joint, and output whether or not the hip joint is abnormal in the target radiographic image by applying the radiographic image to a deep learning model.

10. The hip joint lesion prediction apparatus of claim 9, wherein the number of layers, the number of filters, and the size of the input image of the deep learning model are determined to be interdependent and associated values.

11. The hip joint lesion prediction apparatus of claim 10, wherein the number of layers, the number of filters, and the size of the input image of the deep learning model are determined by values having common exponents and associated with each other.

12. The hip joint lesion prediction apparatus of claim 9, wherein, in the deep learning model, an output of a previous layer is connected to an input of all subsequent layers.

13. The hip joint lesion prediction apparatus of claim 9, wherein, in the deep learning model, a feature map of a previous layer is connected to a feature map of a next layer.

14. The hip joint lesion prediction apparatus of claim 9, wherein the deep learning model comprises one or more non-local blocks for global learning on the input image.

15. The hip joint lesion prediction apparatus of claim 9, wherein the deep learning model is learned based on a hip joint radiographic image classified according to electronic medical records (EMR) medical information extracted using a clinical data warehouse (CDW), and the hip joint radiographic image is labeled with at least one of a normality and a diagnosis.

16. The hip joint lesion prediction apparatus of claim 15, wherein the hip joint radiographic image is preprocessed such that at least one of a character, a symbol, a mark, and a pixel with a preset brightness value or greater included in the hip joint radiographic image is removed.
